(19) United States
(12) Patent Application Publication
(10) Pub. No.: US 2007/0231316 A1
(43) Pub. Date: Oct. 4, 2007

(54) Treatment of Cognitive Dysfunction in Lupus

(76) Inventors: Betty A. Diamond, Bronx, NY (US); Bruce T. Volpe, Bronx, NY (US)

Correspondence Address: AMSTER, ROTHSTEIN & EBENSTEIN LLP, 90 PARK AVENUE, NEW YORK, NY, 10016, US

(21) Appl. No.: 10/574,994

(22) PCT Filed: Oct. 5, 2004
(86) PCT No.: PCT/US04/32675

§ 371 Date: Jan. 31, 2007

Related U.S. Application Data
(60) Provisional application No. 60/509,162, filed on Oct. 7, 2003.

(51) Int. Cl. A61K 38/08; A61K 38/10; A61K 38/16; A61K 39/395

(52) U.S. Cl. 424/130.1; 514/12; 514/13; 514/14; 514/15; 514/16; 514/17

(57) ABSTRACT

Methods of preventing binding of an anti-double stranded (ds)-DNA antibody to a neuron in a mammal exhibiting or at risk for lupus-induced cognitive dysfunction are provided. Methods of inhibiting progression of cognitive dysfunction in a mammal exhibiting or at risk for lupus-induced cognitive dysfunction are also provided. Also provided are methods of inhibiting progression of cognitive dysfunction in a mammal exhibiting or at risk for lupus-induced cognitive dysfunction. Additionally, methods of inhibiting progression of cognitive dysfunction in a mammal exhibiting or at risk for lupus-induced cognitive dysfunction are provided. Further provided are methods of inducing cognitive dysfunction in a mammal, and nonhuman mammal treated with a DNA mimotope in such a manner to induce antibodies that bind to ds-DNA and an NR2 subunit of a neuron in the mammal, then, after the antibodies are induced, treated to temporarily open the blood brain barrier.

### CROSS-REFERENCE TO RELATED APPLICATION

This application claims the benefit of U.S. Provisional Application No. 60/509,162, Filed Oct. 7, 2003.

### BACKGROUND

(1) Field of the Invention

The present invention generally relates to treatments for systemic lupus erythematosus. More particularly, the invention is directed to methods of treating cognitive dysfunction in lupus.

(2) Description of the Related Art

### REFERENCES CITED

Abdel-Rahman, A., A. K. Shetty, M. B. Abou-Donia, Neurobiol Dis 10, 306-26 (August 2002).

The American College of Rheumatology nomenclature and case definitions for neuropsychiatric lupus syndromes. Arthritis Rheum. 42(4): p. 599-608 (1999).

Brey, R. L., et al., Neuropsychiatric syndromes in lupus: prevalence using standardized definitions. Neurology 58(8): p. 1214-20 2002.

Buyon, J. P., et al., Cardiac 5-HT(4) serotoninergic receptors, 52 kD SSA/Ro and autoimmune-associated congenital heart block. J Autoimmun. 19(1-2): p. 79-86 (2002).

Cendes, F., F. Andermann, M. C. Preul, D. L. Arnold, Ann Neurol 35, 211-6 (February, 1994).

Chen, G. et al., Nature 408, 975-9 (Dec. 21-28, 2000).

Cho, S., T. H. Joh, H. H. Baik, C. Dibinis, B. T. Volpe, Brain Res 755, 335-8 (May 2, 1997).

Chu, W. J. et al., Magn Reson Med 43, 359-67 (March, 2000).

Coggeshall, R. E., H. A. Lekan, J Comp Neurol 364, 6-15 (Jan. 1, 1996).

Contet, C., J. N. Rawlins, R. M. Deacon, Behav Brain Res 124, 33-46 (Sep. 28, 2001).

Crawley, J. N., What's Wrong with My Mouse? Behavioral phenotyping of transgenic and knockout mice. (Wiley, N.Y., 2000).

Deacon, R. M., A. Croucher, J. N. Rawlins, Bebav Brain Res 132, 203-13 (May 14, 2002).

DeGiorgio, L. A., et al., A subset of lupus anti-DNA antibodies cross-reacts with the NR2 glutamate receptor in systemic lupus erythematosus. Nat Med. 7(11): p. 1189-93 (2001).

Dellalibera-Joviliano, R., et al., Kinins and cytokines in plasma and cerebrospinal fluid of patients with neuropsychiatric lupus. J Rheumatol. 30(3): p. 485-92 (2003).

Denburg, S. D., J. A. Denburg, in Systemic lupus erythematosus L. R G, Ed. (Academic Press, San Diego, Calif., 1999) pp. 611-629.

Devinsky, O., C. K. Petito, and D. R. Alonso, Clinical and neuropathological findings in systemic lupus erythematosus: the role of vasculitis, heart emboli, and thrombotic thrombocytopenic purpura. Ann Neurol. 23(4): p. 3804 (1988).

Elkon, K. B., E. Bonfa, and N. Brot, Antiribosomal antibodies in systemic lupus erythematosus. Rheum Dis Clin North Am. 18(2): p. 377-90 (1992).

Ellis, S. G. and M. A. Verity, Central nervous system involvement in systemic lupus erythematosus: a review of neuropathologic findings in 57 cases, 1955--1977. Semin Arthritis Rheum. 8(3): p. 212-21 (1979).

Ennaceur, A., J. Delacour, Behav Brain Res 31, 47-59 (Nov. 1, 1988).

Esposito, P. et al., J Pharmacol Exp Ther 303, 1061-6 (December, 2002).

Friedman, A. et al., Nat Med 2, 1382-5 (December, 1996).

Gaynor, B. et al., Proc Natl Acad Sci U S A 94, 1955-60 (Mar. 4, 1997).

Goldstein, F. B., J Biol Chem 244, 4257-60 (Aug. 10, 1969).

Gunderson, H. J. G., Journal of Microscopy 143, 345 (1986).

Hahn, B. H. N Engl J Med 338, 1359-68 (May 7, 1998).

Hahn, B. H., B. Tsao, in Dubois' Lupus Erythematosus D. Wallace, B. H. Hahn, Eds. (Lippincott, Williams & Wilkins, Philadelphia, 2002).

Hanly, J. G., N. M. Walsh, and V. Sangalang, Brain pathology in systemic lupus erythematosus. J Rheumatol. 19(5): p. 73241 (1992).

Hetherington, H. et al., Ann Neurol 38, 396-404 (September, 1995).

Hugg, J. W., K. D. Laxer, G. B. Matson, A. A. Maudsley, M. W. Weiner, Ann Neurol 34, 788-94 (December, 1993).

Hughes, G. R., N. N. Harris, and A. E. Gharavi, The anticardiolipin syndrome. J Rheumatol. 13(3): p. 486-9 (1986).

Irwin, S., Psychopharmacologia 13, 222-57 (Sep. 20, 1968).

John, G. R., S. C. Lee, and C. F. Brosnan, Cytokines: powerful regulators of glial cell activation. Neuroscientist. 9(1): p. 10-22 (2003).

Johnson, R. T. and E. P. Richardson, The neurological manifestations of systemic lupus erythematosus. Medicine (Baltimore). 47(4): p. 337-69 (1968).

Jongen, P. J., et al., Cerebrospinal fluid C3 and C4 indexes in immunological disorders of the central nervous system. Acta Neurol Scand. 101(2): p. 116-21 (2000).

Kao, C. H., et al., The role of FDG-PET, HMPAO-SPET and MRI in the detection of brain involvement in patients with systemic lupus erythematosus. Eur J Nucl Med. 26(2): p. 129-34 (1999).

Kier, A. B., J Comp Pathol 102, 165-77 (February, 1990).

Kozora, E., et al., Magnetic resonance imaging abnormalities and cognitive deficits in systemic lupus erythematosus patients without overt central nervous system disease. Arthritis Rheum. 41(1): p. 41-7 (1998).

Kuzniecky, R. et al., Neurology 48, 1018-24 (April, 1997).

Leritz, E., et al., Neuropsychological functioning and its relationship to antiphospholipid antibodies in patients with systemic lupus erythematosus. J Clin Exp Neuropsychol. 24(4): p. 527-33 (2002).

Leritz, E., J. Brandt, M. Minor, F. Reis-Jensen, M. Petri, J Int Neuropsychol Soc 6, 821-5 (November, 2000).

Lim, M. K., et al., Systemic lupus erythematosus: brain MR imaging and single-voxel hydrogen 1 MR spectroscopy. Radiology 217(1): p. 43-9 (2000).

Morris, R. G., E. Anderson, G. S. Lynch, M. Baudry, Nature 319, 774-6 (Feb. 27-Mar. 5, 1986).

Nomura, K., et al., Asymptomatic cerebrovascular lesions detected by magnetic resonance imaging in patients with systemic lupus erythematosus lacking a history of neuropsychiatric events. Intern Med. 38(10): p. 785-95 (1999).

Nylen, K. et al., J. Neurosci. Res. 67, 844-51 (2002)

Oates, J. C. and G. S. Gilkeson, Mediators of injury in lupus nephritis. Curr Opin Rheumatol. 14(5): p. 498-503 (2002).

Patel, P. and V. Werth, Cutaneous lupus erythematosus: a review. Dermatol Clin. 20(3): p. 373-85 (2002).

Portanova, J. P., et al., Allogeneic MHC antigen requirements for lupus-like autoantibody production and nephritis in murine graft-vs-host disease. J Immunol. 141(10): p. 3370-6 (1988).

Putterman, C., B. Diamond, J Exp Med 188, 29-38 (Jul. 6, 1998).

Sabet, A., et al., Neurometabolite markers of cerebral injury in the antiphospholipid antibody syndrome of systemic lupus erythematosus. Stroke. 29(11): p. 2254-60 (1998).

Sakimura, K. et al., Nature 373, 151-5 (Jan. 12, 1995).

Sanna, G., et al., Central nervous system involvement in systemic lupus erythematosus: cerebral imaging and serological profile in patients with and without overt neuropsychiatric manifestations. Lupus. 9(8): p. 573-83 (2000).

Sharma, A., D. Isenberg, B. Diamond, Rheumatology (Oxford) 42, 453-63 (March, 2003).

Sibbitt, W. L., Jr., R. R. Sibbitt, and W. M. Brooks, Neuroimaging in neuropsychiatric systemic lupus erythematosus. Arthritis Rheum. 42(10): p. 2026-38 (1999).

Tsien, J. Z., P. T. Huerta, S. Tonegawa, Cell 87, 1327-38 (Dec. 27, 1996).

Urenjak, J., S. R. Williams, D. G. Gadian, M. Noble, J Neurochem 59, 55-61 (July, 1992).

Volpe, B. T., J. Wildmann, C. A. Altar, Neuroscience 83, 741-8 (April, 1998).

Weiner, S. M., et al., Diagnosis and monitoring of central nervous system involvement in systemic lupus erythematosus: value of F-18 fluorodeoxyglucose PET. Ann Rheum Dis. 59(5): p. 377-85 (2000).

Winfield, J. B., I. Faiferman, D. Koffler, J Clin Invest 59, 90-6 (January, 1977).

Xaio, H., W. A. Banks, M. L. Niehoff, J. E. Morley, Brain Res 896, 36-42 (Mar. 30, 2001).

Systemic lupus erythematosus is a disease characterized by the production of autoantibodies, especially anti-nuclear antibodies. It is clear that these antibodies contribute to several disease manifestations. Autoantibodies can mediate renal damage, hematologic abnormalities, serositis, skin disease and thrombosis (Hahn and Tsao, 2002). The antigenic specificities of the pathogenic autoantibodies continue to be determined, but antibodies that bind to ds-DNA are present in affected glomeruli, skin, and serosal surfaces in lupus patients (Winfield et al., 1977; Hahn, 1998).

As patients with SLE live longer, clinicians have noted that many experience central 20 nervous system (CNS) manifestations of disease. That SLE might underlie a general assault on the central nervous system (CNS) is consistent with a growing number of clinical, pathological and neuro-imaging studies. The Neuropsychiatric Lupus Nomenclature Comrnittee (American College of Rheumatology, 1999) identified 19 different symptom complexes within the diagnosis of neuropsychiatric lupus (NP-SLE). For some of the categories on this comprehensive list, there are solid candidate causative mechanisms. For others, especially the psychiatric and cognitive disorders, there is less information about mechanism or pathology. No wonder then that a single all-purpose unifying mechanism for NP-SLE has remained elusive.

SLE is characterized by an activation of the immune system leading to autoantibody production, engagement of complement cascades and exuberant cytokine production, all perhaps under the permissive effect of hormones. It is now well established that autoantibodies can directly damage organs, especially the kidney (Portanova et al., 1988; Oates and Gilkeson, 2002), skin (Patel and Werth), and fetal heart (Buyon et al., 2002). There is provocative data suggesting that autoantibodies may also mediate brain damage. In fact, there is a long history of efforts to link autoantibodies in SLE directly to CNS manifestations of disease. Several investigators have sought to identify autoantibodies that either bind directly to neurons or, at high titer, associate with NP SLE. One autoantibody specificity for which there is a plausible mechanistic explanation for a relationship to brain dysfunction is anti-phospholipid antibodies (Hughes et al., 1986; Lenitz et al., 2002). These pathological autoantibodies impair the rheological conditions in large and small vessels to generate emboli and thrombosis that cause ischemic and hemorrhagic brain injury. Anti-ribosomal P protein autoantibodies have been correlated in some, but not all, studies with psychosis (Elkon et al., 1992). Recently, antibodies to the NR2 NMDA receptor were demonstrated to be present in lupus serum and CSF and to have the functional capacity to cause excitotoxic neuronal death (DeGiorgio et al., 2001). Some neuropathological studies of patients with NP-SLE have implicated complement (Hanly et al., 1992), and recent studies of cerebrospinal fluid C3 and C4 indices have demonstrated increased values for patients with NP-SLE consistent with a potential for antibody mediated activation of the complement cascade (Jongen et al., 2000). Autoantibodies are not, however, the only potential causative agent of CNS damage. A growing literature of cytokine abnormalities in the CSF of lupus patients is matched by the complementary findings that cytokines can directly affect neuronal survival and function (Dellalibera-Joviliano et al., 2003; John et al., 2003). Finally, brain function may also suffer from the considerable secondary effects from systemic organ failure and from direct toxicity of the treatments for SLE.

There is some consensus that cognitive dysfunction, one of the 19 syndromic complexes included in NP-SLE, may be emerging as a frequent and debilitating manifestation of NP-SLE. Four major neuropathological analyses of 141 patients with all varieties of NP-SLE have provided some clues to potential mechanisms for the non-reversible cognitive impairment. Cerebrovascular disease with destructive and proliferative changes in large vessels or in small vessels with multiple small infarctions occurred in 66 patients; vasculitis occurred rarely in only 11 (Hanly et al., 1992; Devinskh egt al., 1988; Johnson and Richardson, 1968; Ellis and Verity, 1979). Ischemic or hemorrhagic brain injury may underlie the non-reversible cognitive disorder, and, when progressive or cumulative, may cause atrophy and multi-infarct dementia, yet many patients with cognitive impairments have no demonstrable vascular lesion.

Neuroimaging studies have offered new opportunities to explore brain metabolism and structural lesions in vivo and can be informative even in individuals with subtle neurological disorders. Recent imaging studies in SLE confirm that stroke correlates with the presence of antiphoshoplipid antibodies. Many abnormalities have been noted with magnetic resonance imaging, magnetic resonance spectroscopy and positron emission tomographic techniques. There has, however, been no consistent structural or metabolic abnormality in imaging studies that is correlated with cognitive impairment (Kao et al, 1999; Kozora et al., 1998; Lim et al., 2000; Nomura et al., 1999; Sabet et al., 1998; Sanna et al., 2000; Sibbitt et al., 1999; Brey et al., 2002).

A recent study using ACR diagnostic criteria and reliable neuropsychological batteries demonstrated a prevalence of NP-SLE of 80% (Brey et al., 2002). Consistent with past work, these investigators show that nearly 50% of patients had mild to severe cognitive impairment, and this impairment was particularly significant in the areas of motor performance speed, and visual and verbal memory. This observation suggests that a major component of cognitive impairment may occur initially with regional brain injury, and that impairment might continue to accrue over time from the cumulative more general vasculopathic or toxic brain damage. It is reasonable, therefore, to speculate that a longitudinal, in addition to a cross sectional, approach might efficiently reveal mechanisms that underlie cognitive impairment. The formal assessment of behavior along with concomitant neuroimaging of metabolic and structural brain damage in the context of measurement of disease activity and serum autoantibody levels may provide new insights into disease mechanisms.

Using immunochemistry to identify the light subunit of the neurofilament triplet protein (NFL) and the glial fibrillary acidic protein (GFAP) in the cerebrospinal fluid (CSF) of patients, Nylen et al. (2002) argue for new, sensitive surrogate markers for the complex but increasingly diagnosed brain disorder that occurs in patients with SLE, NP-SLE. Finding the filamentous components of the cytoskeleton of neurons and astrocytes in the CSF suggests that neurons are dying and implicates astrocytes in the pathology. Although neither measure can be considered diagnostic for a specific disease, this work by Nylen et al. demonstrates unequivocally that some patients with SLE sustain persistent central nervous system injury. They demonstrate a logarithmic increase in NFL and a significant increase GFAP levels in SLE patients with brain dysfunction compared to SLE patients without evidence of CNS disease and to normals. They show that an increase in these markers correlated with inflammatory cytokines and oligoclonal bands in the CSF, and structural changes on the magnetic resonance brain images. Further, in a smaller group of NP-SLE patients, they show a marked decline of the NFL and GFAP levels after cyclophosphamide treatment.

Thus, there is a need for studies to determine causes of cognitive dysfunction in SLE, and development of treatments based on those causes. The present invention satisfies that need.

### SUMMARY OF THE INVENTION

Accordingly, the inventors have discovered that a cause for cognitive dysfunction in SLE is the entry across the blood-brain barrier of anti-dsDNA antibodies that bind to the NR2 subunit of neuronal NMDA receptors, and the subsequent apoptosis of those neurons in the brain, particularly the hippocampus. Methods and compositions related to this discovery are provided.

Thus, in some embodiments, the invention is directed to methods of preventing binding of an anti-double stranded (ds)-DNA antibody to a neuron in a mammal exhibiting or at risk for lupus-induced cognitive dysfunction. The anti-ds-DNA antibody in this method binds to an NR2 subunit of an NMDA receptor on the neuron. The methods comprise treating the mammal with at least one peptide or mimetic in an amount effective to bind to the antibody, where the peptide or mimetic comprises an amino acid sequence of X1-Trp-X1-Tyr-X2, wherein X1 represents Asp or Glu, and X2 represents Gly or Ser.

In other embodiments, the present invention is directed to methods of inhibiting progression of cognitive dysfunction in a mammal exhibiting or at risk for lupus-induced cognitive dysfunction. The methods comprise treating the mammal with at least one peptide or mimetic in an amount effective to bind to anti-ds-DNA antibodies that bind to an NR2 subunit of an NMDA receptor on a neuron. In these methods, the peptide or mimetic comprises an amino acid sequence of X1-Trp-X1-Tyr-X2, where X1 represents Asp or Glu, and X2 represents Gly or Ser.

The invention is also directed to methods of inhibiting progression of cognitive dysfunction in a mammal exhibiting or at risk for lupus-induced cognitive dysfunction. The methods comprise treating the brain of the mammal with an agent that prevents binding of an anti-ds-DNA antibody to an NR2 subunit of an NMDA receptor of a neuron.

In related embodiments, the invention is additionally directed to methods of inhibiting progression of cognitive dysfunction in a mammal exhibiting or at risk for lupus-induced cognitive dysfunction. The methods comprise treating the brain of the mammal with an agent that inhibits death of a neuron that comprises a bound anti-ds-DNA antibody on NR2 subunits of an NMDA receptor on the neuron.

The invention is further directed to methods of inducing cognitive dysfunction in a mammal. The methods comprise treating the mammal with a DNA mimotope in such a manner to induce antibodies that bind to ds-DNA and an NR2 subunit of a neuron in the mammal, then, after the antibodies are induced, treat the mammal to temporarily open the blood brain barrier.

In related embodiments, the invention is also directed to a nonhuman mammal treated with a DNA mimotope in such a manner to induce antibodies that bind to ds-DNA and an NR2 subunit of a neuron in the mammal, then, after the antibodies are induced, treated to temporarily open the blood brain barrier.

### BRIEF DESCRIPTION OF THE DRAWINGS

FIG. 1 is two graphs establishing that mice immunized with MAP-peptide produce anti-peptide, anti-DNA antibodies. The graphs show titers of antibody to (A) DWEYS or (B) dsDNA in serum of mice immunized with MAP-DWEYS or MAP-Core (polylysine backbone). Assays were performed on serum obtained at week nine after the first immunization. Ten mice were included in each group.

FIG. 2 shows light and fluorescent micrographs and a graph showing that anti-NR2 antibodies, in conjunction with an opening of the blood brain barrier, lead to neuronal damage. Animals immunized with MAP-peptide receiving no LPS treatment displayed normal hippocampal histology. Panel A is a light micrograph of a coronal section from one of four animals through the dorsal hippocampus after staining with cresyl violet (pyramidal neurons in CA1 region stained blue, magnification 5×). The asterisk denotes the region depicted in the inset (40×). Sections from all four animals were similarly without pathology. Panel B is a micrograph of the same section showing no immunoglobulin deposition present. Panel C shows a brain section of MAP-core immunized mouse seven days after LPS exposure. Brain sections displayed diffuse IgG deposition. A representative coronal section of the hippocampus is shown. Panel D shows a representative coronal section of the hippocampus of four animals treated with LPS after MAP-peptide immunization. IgG deposition was intensely concentrated in neurons in the CA1 region of the hippocampus. Panels E and F shows activated caspase-3 (Panel E) and Fluoro-Jade immunofluoresence (Panel F) were present in the CA1 region of the hippocampus in animals immunized with MAP-peptide and treated with LPS (40×). There was no immunofluorescent reactivity in animals immunized with MAP-core and treated with LPS (data not shown). Panel G is a graph showing the results of quantitative morphometric analysis in LPS treated MAP-peptide and MAP-core immunized animals after a seven day (N=8) and thirty day (N=10) survival, which demonstrates that MAP-peptide immunized animals sustained a significant loss of CA1 hippocampal neurons (P<0.0001 for both groups). Neuron number is expressed as the density of neuron/105 microns3 (modified from Cho et al., 1997; Volpe et al., 1998). Hippocampal volumes were comparable (P=0.56).

FIG. 3 is magnetic resonance spectroscopy data showing an altered NAA/Cr ratio in MAP-peptide immunized LPS treated mice. Panel A is a graph showing hippocampal NAA/Cr ratios for three MAP-peptide and MAP-core treated mice. Values represent mean ±s.d. There is a significant reduction in NAA/CR in the hippocampus of the MAP-peptide immunized LPS treated mice in comparison to the MAP-core immunized, LPS treated mice (P<0.005). Panel B shows a T1 weighted anatomical image of a MAP-peptide immunized mouse on the left, and the spectroscopic imaging grid identifying the hippocampus from a MAP-core immunized mouse on the right. A representative spectrum from the hippocampus is shown below. Panel C depicts the spectra from the hippocampus and thalamus from a MAP-peptide immunized mouse and MAP-core immunized mouse.

FIG. 4 is graphs of experimental data demonstrating that mice with anti-NR2 antibodies and given LPS develop cognitive dysfunction. Panel A shows that mice immunized with MAP-peptide and given LPS (MAP-peptide) had comparable motor balance, as demonstrated by the accelerating rotarod (4-40 rpm) test, compared to mice immunized with MAP-core given LPS (MAP-Core). Mice underwent 3 consecutive sessions (maximum, 5 min). Panel B shows that MAP-peptide treated mice displayed normal anxiety-related parameters, as observed in the elevated plus maze (at left, parameters measured: latency to enter the open arms, entries into open arms and time spent in them) and in the black-white alley (at right, parameters measured: latency to enter white area, crossings between alleys, and the time spent in the white alley). Panel C shows that MAP-peptide treated mice showed normal object-recognition memory. In the sample phase, mice explored two identical objects. After a delay, in the test phase, mice explored a familiar object (similar to the ones in the sample phase) and a novel object. The exploration ratios (E1 and E2), preference index and discrimination ratio (see Methods for definitions) revealed no differences between groups. Panel D shows that MAP-peptide treated mice showed increased locomotion in a novel area (left), revealed as significantly more squares crossed (MAP-peptide, 10.3±0.9; MAP-core, 4.2±0.5 squares crossed, P=0.0000025). At right, MAP-peptide treated mice spent more time moving during repeated 5 minute exposures to an arena (session 3, MAP-peptide, 141.6±16.5; MAP-core, 101.5±8.1 sec, P=0.029). Panel E shows that MAP-peptide treated mice were impaired on a spatial working memory task in the t-maze. Animals performed roughly at chance throughout the test (MAP-peptide, 52.6±4.0, MAP-core, 71.7±4.2% alternations during all trials, P=0.0027). Panel F shows spatial reference memory assessment in the Morris water maze. Mice underwent 12 trials with a large hidden platform (diameter, 24-cm), followed by a probe test (Probe 1). Thereafter, mice underwent 24 trials with a small hidden platform (diameter, 16 cm), followed by Probe 2. The left graph shows the latency to reach the hidden platform (maximum search time, 90 sec), whereas the right graph shows the time spent in each sector during the probe trials. MAP-peptide immunized mice displayed reduced bias to the former target, an indication of memory deterioration (Probe 2, MAP-peptide, 35.1±3.5%, MAP-core, 48.4±3.8% time; P=0.018). Abbreviations for sectors: T, target, O, opposite, L, left, R, right. Panel G shows that MAP-peptide treated mice were impaired in the training-to-criterion task. The left graphic shows the scheme of the task: if a mouse found the hidden platform in location N in less than 20 sec for 3 consecutive runs, then a new location (N+1) was selected. Mice were trained to criterion on 5 consecutive locations. The right graphic shows that Exp mice were significantly poorer (MAP-peptide, 12.6±1.2, MAP-core, 6.9±0.4 trials-to-criterion [inclusive], P=0.0000017) in their ability to learn a given location.

FIG. 5 is micrographs of a post-mortem brain section from a lupus patient immunostained for IgG.

FIG. 6 is micrographs comparing staining of the same section of a post-mortem brain of a lupus patient for IgG or NR2. The graphic representation shows that the patient's own antibody binds in the same place as the anti-NR2 antibody. Hence, it is highly suggestive that the patient's antibody is binding NR2.

### DETAILED DESCRIPTION OF THE INVENTION

The present invention is based on the discovery that a cause for cognitive dysfunction in SLE is the entry across the blood-brain barrier of anti-dsDNA antibodies that bind to the NR2 subunit of neuronal NMDA receptors, and the subsequent apoptosis of those neurons in the brain, particularly the hippocampus. Experiments establishing this discovery are provided in Example 1 and further confirmed in Example 2.

Thus, in some embodiments, the invention is directed to methods of preventing binding of an anti-double stranded (ds)-DNA antibody to a neuron in a mammal exhibiting or at risk for lupus-induced cognitive dysfunction. In these embodiments, the anti-ds-DNA antibody binds to an NR2 subunit of an NMDA receptor on the neuron. The methods comprise treating the mammal with at least one peptide or mimetic in an amount effective to bind to the antibody, where the peptide or mimetic comprises an amino acid sequence of X1-Trp-X1-Tyr-X2, wherein X1 represents Asp or Glu, and X2 represents Gly or Ser.

As is well known, the use of a peptide mimetic, also known as peptidomimetic, can provide resistance against protease degradation. When discussing a peptide or mimetic, the term “amino acid” includes the mimetic analog of an amino acid. Several types of peptide mimetics are known, including D-amino acids, and peptides that are chemically modified. In preferred embodiments, the peptide or mimetic useful for the present invention comprises D-amino acids.

The peptide or mimetic can be any length, provided the X1-Trp-X1-Tyr-X2 moiety is sufficiently exposed to bind to the anti-ds-DNA antibody binding site. In preferred embodiments, the peptide or mimetic is 5-30 amino acids in length. In more preferred embodiments, the peptide or mimetic is 5-10 amino acids in length. In the most preferred embodiments, the peptide or mimetic is 5 amino acids in length. The most preferred peptide or mimetic comprises Asp-Trp-lu-Tyr-Ser.

These methods can be used with any mammal, including rodents. In preferred embodiments, the mammal is a human.

The most effective administration of the peptide or mimetic for any application can be made without undue experimentation. It is expected that timely and sufficient administration of the peptide or mimetic as an oral, lingual, sublingual, buccal, intrabuccal, or parenteral administration would block anti-ds-DNA antibodies from binding to neuronal NR2 subunits sufficiently to prevent cognitive dysfunction if the antibodies pass the blood-brain barrier, however, direct administration to the brain, preferably directly to the hippocampus, e.g., by injection, would be expected to be more directly effective, particularly if the anti-ds-DNA antibodies have already passed the blood-brain barrier, i.e., in the cerebrospinal fluid.

The above-described peptide or mimetic compositions can be formulated without undue experimentation for administration to a mammal, including humans, as appropriate for the particular application. Additionally, proper dosages of the compositions can be determined without undue experimentation using standard dose-response protocols.

Accordingly, the peptide or mimetic compositions designed for oral, lingual, sublingual, buccal and intrabuccal administration can be made without undue experimentation by means well known in the art, for example with an inert diluent or with an edible carrier. The compositions may be enclosed in gelatin capsules or compressed into tablets. For the purpose of oral therapeutic administration, the pharmaceutical compositions of the present invention may be incorporated with excipients and used in the form of tablets, troches, capsules, elixirs, suspensions, syrups, wafers, chewing gums and the like.

Tablets, pills, capsules, troches and the like may also contain binders, recipients, disintegrating agent, lubricants, sweetening agents, and flavoring agents. Some examples of binders include microcrystalline cellulose, gum tragacanth or gelatin. Examples of excipients include starch or lactose. Some examples of disintegrating agents include alginic acid, corn starch and the like. Examples of lubricants include magnesium stearate or potassium stearate. An example of a glidant is colloidal silicon dioxide. Some examples of sweetening agents include sucrose, saccharin and the like. Examples of flavoring agents include peppermint, methyl salicylate, orange flavoring and the like. Materials used in preparing these various compositions should be pharmaceutically pure and nontoxic in the amounts used.

The peptide or mimetic compositions of the present invention can easily be administered parenterally such as for example, by intravenous, intramuscular, intrathecal or subcutaneous injection. Parenteral administration can be accomplished by incorporating the compositions of the present invention into a solution or suspension. Such solutions or suspensions may also include sterile diluents such as water for injection, saline solution, fixed oils, polyethylene glycols, glycerine, propylene glycol or other synthetic solvents. Parenteral formulations may also include antibacterial agents such as for example, benzyl alcohol or methyl parabens, antioxidants such as for example, ascorbic acid or sodium bisulfite and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials made of glass or plastic.

Rectal administration includes administering the pharmaceutical peptide or mimetic compositions into the rectum or large intestine. This can be accomplished using suppositories or enemas. Suppository formulations can easily be made by methods known in the art. For example, suppository formulations can be prepared by heating glycerin to about 120° C., dissolving the composition in the glycerin, mixing the heated glycerin after which purified water may be added, and pouring the hot mixture into a suppository mold.

Transdermal administration includes percutaneous absorption of the composition through the skin. Transdermal formulations include patches (such as the well-known nicotine patch), ointments, creams, gels, salves and the like.

The present invention includes nasally administering to the mammal a therapeutically effective amount of the peptide or mimetic composition. As used herein, nasally administering or nasal administration includes administering the composition to the mucous membranes of the nasal passage or nasal cavity of the patient. As used herein, pharmaceutical compositions for nasal administration of a composition include therapeutically effective amounts of the composition prepared by well-known methods to be administered, for example, as a nasal spray, nasal drop, suspension, gel, ointment, cream or powder. Administration of the composition may also take place using a nasal tampon or nasal sponge.

The effectiveness of the treatment can be monitored using any method known in the art, and the skilled artisan can establish a useful monitoring method for any particular application without undue experimentation. Non-limiting examples include methods for quantifying the amount of antibody binding to the brain, neurons, hippocampal neurons, or NR2 subunits; testing cognitive dysfunction, using any of several well-known tests; or imaging techniques for evaluating neuronal degeneration, such as magnetic resonance spectroscopic imaging. It is believed that the latter method is preferred in many situations, since the direct cause of cognitive dysfunction, neuronal degeneration, can be observed.

As established in the example, the most relevant neurons to be protected to prevent, or inhibit the progression of, cognitive dysfunction are hippocampal neurons. Thus, the most effective treatments will inhibit binding of anti-ds-DNA antibodies and/or progression of neuronal degeneration in the hippocampus.

In other embodiments, the present invention is directed to methods of inhibiting progression of cognitive dysfunction in a mammal exhibiting or at risk for lupus-induced cognitive dysfunction. The methods comprise treating the mammal with at least one peptide or mimetic in an amount effective to bind to anti-ds-DNA antibodies that bind to an NR2 subunit of an NMDA receptor on a neuron. In these embodiments, as in the embodiments described above, the peptide or mimetic comprises an amino acid sequence of X1-Trp-X1-Tyr-X2, wherein X1 represents Asp or Glu, and X2 represents Gly or Ser.

As in the above-described embodiments, the mimetic can be any mimetic known in the art, but preferably comprises D-amino acids. The peptide or mimetic can also be any length, but is preferably 5-30 amino acids, more preferably 5-10 amino acids, and most preferably 5 amino acids in length. The most preferred peptide or mimetic comprises Asp-Trp-Glu-Tyr-Ser.

These methods also can be used with any mammal, including rodents, but is most useful for a human, as with embodiments described above. The most useful neuron to be targeted to inhibit cognitive dysfunction is a hippocampal neuron, although the peptide will inhibit binding of the anti-ds-DNA antibodies to any neuron. As is well-known, the NMDA receptor is present in all neurons. The method would therefore be expected to protect any neuron from excitatory cell death due to binding of the anti-ds-DNA binding to the NR2 subunit of the NMDA receptor.

The effectiveness of these embodiments can be monitored by any appropriate means, preferably with magnetic resonance spectroscopic imaging or behavioral tests of hippocampal-dependent performance, such as a memory test.

These methods can be preceded with a determination of the risk for lupus-induced cognitive dysfunction, preferably by determining whether the mammal has anti-NR2 antibodies, where the presence of anti-NR2 antibodies indicates that the mammal is at risk for lupus-induced cognitive dysfunction. This determination preferably includes a test to determine whether the anti-NR2 antibodies have crossed the blood-brain barrier, preferably by testing the cerebrospinal fluid for the presence of anti-NR2 antibodies. These methods are not narrowly limited to any particular test for anti-NR2 antibodies, as several suitable tests are known. A preferred example is the ELISA described in the Example, where binding of antibodies to the DWEYS peptide is assayed.

The present invention is also directed to methods of inhibiting progression of cognitive dysfunction in a mammal exhibiting or at risk for lupus-induced cognitive dysfunction. The methods comprise treating the brain of the mammal with an agent that prevents binding of an anti-ds-DNA antibody to an NR2 subunit of an NMDA receptor of a neuron. These methods include treatment of the brain of the mammal with agents that bind to the antibody, preventing binding to the NR2 subunit, as well as agents that bind to the NMDA receptor, particularly the NR2 subunit, blocking the subunit from binding to the anti-ds-DNA antibody.

As used herein, “treating the brain of the mammal with an agent” includes treatment of the mammal outside the brain (e.g., parenterally) with an agent that can cross the blood-brain barrier, as well as directly applying the agent to the brain.

Non-limiting examples of useful agents include an antibody or an aptamer that binds to the NMDA receptor on a neuron but does not induce neuronal death.

In preferred embodiments, the neuron that is blocked from anti-ds-DNA antibody binding is a hippocampal neuron, since death of hippocampal neurons are the apparent direct cause of lupus cognitive dysfunction.

In further embodiments, the invention is also directed to methods of inhibiting progression of cognitive dysfunction in a mammal exhibiting or at risk for lupus-induced cognitive dysfunction. The methods comprise treating the brain of the mammal with an agent that inhibits death of a neuron that comprises a bound anti-ds-DNA antibody on NR2 subunits of an NMDA receptor on the neuron. The rationale behind these methods is to protect the neurons of the mammal from the effects of anti-ds-DNA binding to the NR2 subunit, rather than inhibiting the binding itself. These methods are particularly useful due to their non-specificity, since they protect the neurons from excitatory death regardless of the cause.

As with previous embodiments, it is particularly preferred in these embodiments that the hippocampus is treated, since death of hippocampal neurons is the apparent cause of cognitive dysfunction in lupus.

As is known in the art, the various stages of excitatory neuronal death suggests several areas where neuronal death can be inhibited. In one aspect, oxidative stress can be reduced in the neuron, e.g., with antioxidants, superoxide reductants such as superoxide dismutase (SOD) or SOD mimetics, several of which are known in the art. Additionally, glutathione levels in the neuron can be increased by various known methods. Other approaches include the use of an agent that induces transcription factors that prevent apoptosis of the neuron, and/or an agent that inhibits transcription factors that induce apoptosis of the neuron; agents that increase aerobic glycolysis in mitochondria of the neuron can also be employed.

In additional embodiments, the invention is directed to methods of inducing cognitive dysfunction in a nonhuman mammal. The methods comprise treating the mammal with a DNA mimotope in such a manner to induce antibodies that bind to ds-DNA and an NR2 subunit of a neuron in the mammal, then, after the antibodies are induced, treat the mammal to temporarily open the blood brain barrier. See Example for a preferred way of executing these methods.

These embodiments are useful as a model for lupus-induced cognitive dysfunction, as well as any dysfunction caused by neuronal death. Thus, this model can be used to test for compounds that improve cognitive dysfunction, or reduce excitotoxic neuronal death.

In preferred embodiments, the DNA mimotope is an octamer on a polylysine backbone comprising the peptide or mimetic comprising the sequence X1-Trp-X1-Tyr-X2, wherein X1 represents Asp or Glu, and X2 represents Gly or Ser. As described in the Example, this DNA mimotope induces anti-ds-DNA antibodies that bind to the NR2 subunit of an NMDA receptor, which is present on all neurons. More preferably, the peptide or mimetic comprises the sequence Asp-Trp-Glu-Tyr-Ser. In the most preferred embodiments, the peptide or mimetic consists of the sequence Asp-Trp-Glu-Tyr-Ser.

The blood-brain barrier in these methods can be temporarily opened by any known means. The preferred method is by treatment with lipopolysaccharide (LPS). See Example.

This method could be used in any nonhuman mammal. In preferred embodiments, the mammal is a mouse.

In related embodiments, the invention is directed to a nonhuman mammal treated with a DNA mimotope in such a manner to induce antibodies that bind to ds-DNA and an NR2 subunit of a neuron in the mammal, then, after the antibodies are induced, treated to temporarily open the blood brain barrier.

This mammal exhibits cognitive dysfunction and thus can serve as a useful model, as discussed previously.

In preferred embodiments, the DNA mimotope is an octamer on a polylysine backbone comprising the peptide or mimetic comprising the sequence X1-Trp-X1-Tyr-X2, wherein X1 represents Asp or Glu, and X2 represents Gly or Ser. More preferably, the peptide or mimetic comprises the sequence Asp-Trp-Glu-Tyr-Ser. In the most preferred embodiments, the peptide or mimetic consists of the sequence Asp-Trp-Glu-Tyr-Ser.

The blood-brain barrier of these mammals can be breached by any known method. Preferably, the barrier is breached with lipopolysaccharide (LPS), as disclosed in the Example.

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### EXAMPLE 1

### Example Summary

Patients with systemic lupus erythematosus (SLE) may experience progressive cognitive disabilities. We previously demonstrated that SLE patients produce anti-DNA antibodies that cross-react with the NR2a and 2b subunits of the NMDA receptors, and that these antibodies can mediate excitotoxic death of neurons. We now demonstrate that mice producing anti-NR2 antibodies have no neuronal damage until there is a breakdown of the blood-brain barrier. Once the antibodies gain access to the brain, they bind preferentially to hippocampal neurons, and cause neuronal death with resulting cognitive dysfunction and associated abnormalities in magnetic resonance spectroscopy. This study provides a model for systemic immune responses in SLE leading to cognitive impairment.

Introduction

Studies of brain pathology in lupus-prone strains of mice are difficult because the mice make a broad spectrum of autoantibodies and have infiltrating inflammatory cells in the brain (Kier, 1990). We have demonstrated that BALB/c mice immunized with the DNA mimotope arrayed as an octamer on a polylysine backbone (MAP-peptide) develop cross-reactive anti-double stranded DNA and anti-NR2 antibodies (Gaynor et al., 1997; Putterman and Diamond, 1998). This model affords the opportunity to examine whether, and under what conditions, these antibodies might cause brain pathology and cognitive dysfunction. Because BALB/c mice do not spontaneously develop brain pathology, we could examine the effect of a systemic antibody response without confounding causes of brain damage.

Results

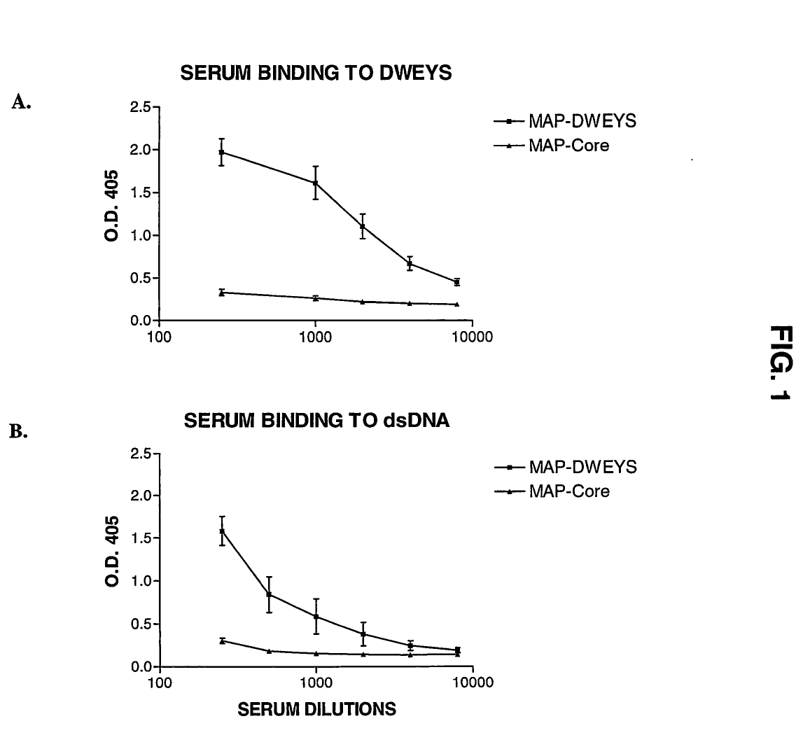

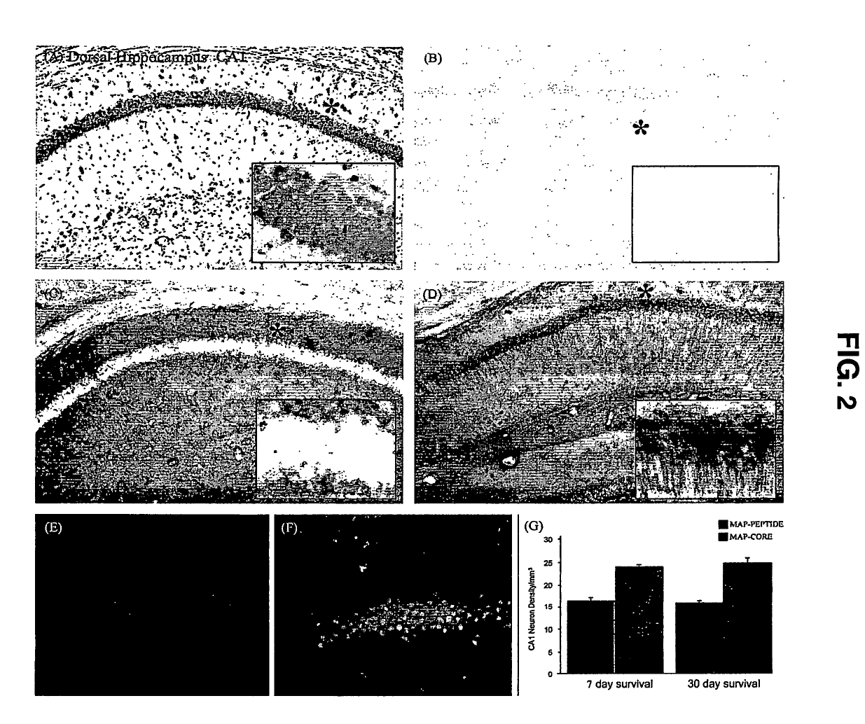

Anti-NR2 antibodies cause brain injury only when there is an abrogation of the blood brain barrier. BALB/c mice were immunized with MAP-peptide or with MAP-core (polylysine backbone alone), as previously described (Putterman and Diamond, 1998). All mice immunized with MAP-peptide developed high titered anti-peptide antibodies and anti-DNA antibodies (FIG. 1), but histopathological examination of their brains revealed no neuronal damage, and no immunoglobulin deposition (FIGS. 2A and B). The most plausible explanation for the absence of brain pathology was an intact blood-brain barrier, preventing the transport of antibody from the systemic circulation into the brain. It has previously been shown that lipopolysaccharide (LPS) can cause a temporary opening of the blood-brain barrier (Xaio et al., 2001). BALB/c mice immunized with either MAP-peptide or MAP-core were given LPS in two injections over 24 hours. Seven days later, those MAP-core immunized mice lacking serum titers of anti-NR2 antibody displayed diffuse IgG deposition in the brain (FIG. 2C), while MAP-peptide immunized mice with anti-NR2 antibody showed intense IgG deposition in hippocampal neurons where there is a high density of NR2 expression (FIG. 2D). There was no complement deposition (data not shown), and no cellular infiltrate in the brains of either group of mice. No blood derived inflammatory cells were present and there was no recruitment of microglial cells or astrocytes to regions of IgG deposition.

To determine whether there was evidence for neuronal damage, brain sections were stained with antibody to activated caspase 3, which identifies neurons in a late stage of apoptosis, and with fluorojade, which identifies pre-apoptotic neurons. Brain sections from mice harboring specific anti-NR2 antibody and displaying IgG deposition demonstrated neurons that were immuno-reactive for activated caspase and fluorojade (FIGS. 2E and F). Damage was highly selective for hippocampal neurons; there was no evidence of damage in regions free of IgG deposition. There was no evidence of neuronal damage in the brains of mice that received LPS but harbored no anti-NR2 antibodies in their serum. By seven days following LPS exposure, mice displayed a 30% loss of pyramidal neurons in CA1 region of the hippocampus (FIG. 2G).

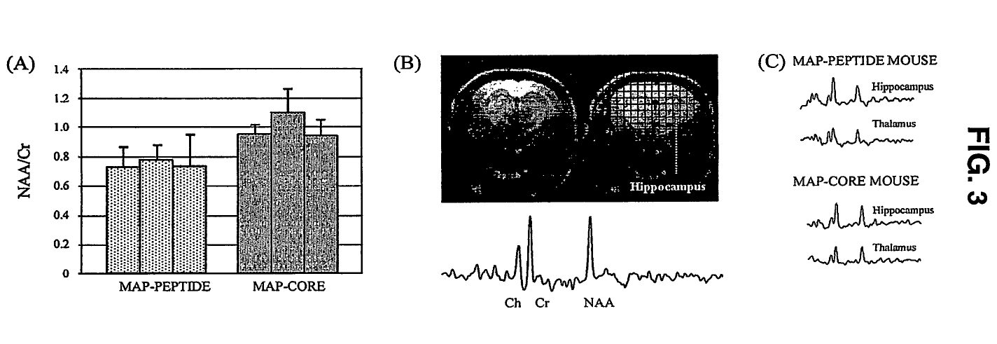

Magnetic resonance spectroscopy provides in vivo confirmation of neuronal dysfunction. Because brain biopsies are potentially hazardous, there has been a recent focused effort to develop techniques for imaging the brain in vivo. Magnetic resonance imaging of the brain provides evidence for structural lesions, but magnetic resonance spectroscopic imaging provides images of major brain metabolites, including N-acetyl aspartate (NAA), which is found only in neurons (Urenjak et al., 1992) and synthesized only in neuronal mitochondria (Goldstein, 1969). NAA, and the ratio of NAA/Creatine (NAA/Cr) has been extensively used in the human brain to identify regions of neuronal loss/damage in patients with epilepsy (Hugg et al., 1993; Hetherington et al., 1995; Cendes et al., 1994) in the presence or absence detectable neuronal loss measured by MRI (Chu et al., 2000; Kuzniecky et al., 1997). Mice immunized with MAP-peptide or with MAP-core and treated with LPS were examined thirty days after LPS exposure and after behavioral assessment. Both groups of mice displayed no structural lesion by MRI (data not shown, and had comparable hippocampal volume by quantitative morphology). Those mice with anti-NR2 antibody displayed decreased hippocampal N-acetyl aspartate NAA/Cr ratios (FIG. 3A). Representative spectra and images from a control mouse are shown in FIG. 3B along with comparison spectra in FIG. 3C. Thus, it was possible to obtain in vivo documentation of neuronal loss/damage in this model.

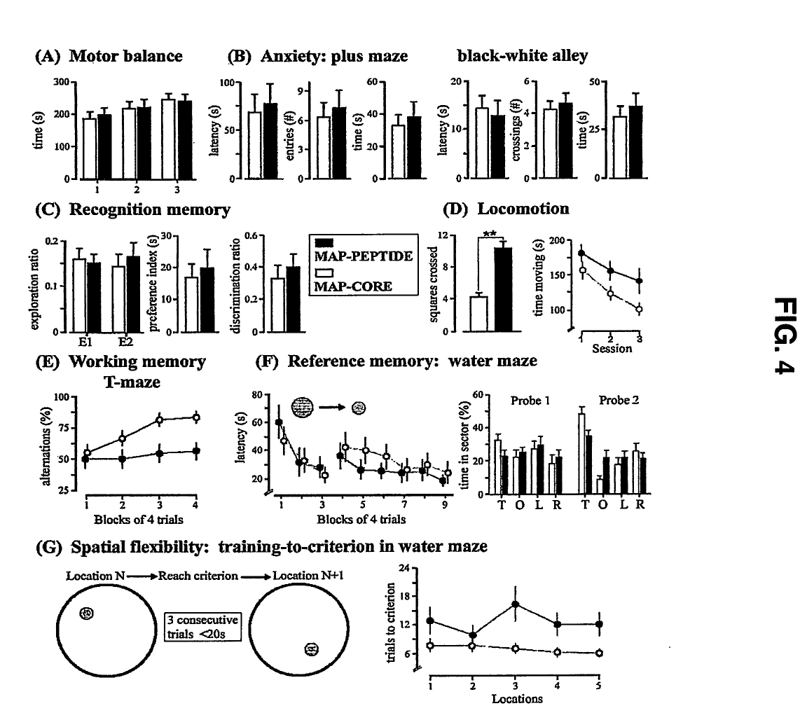

Affected mice display poor memory function. The hippocampus and the function of NMDA receptors are crucial in learning and memory (Tsien et al., 1996; Sakimura et al., 1995). Since we were able to demonstrate a selective loss of hippocampal neurons and a selective alteration in hippocampal NAA/Cr ratios in mice with both anti-NR2 antibody and a loss of integrity of the blood-brain barrier, we examined whether these mice displayed a behavioral phenotype, particularly in hippocampal-dependent performance. MAP-peptide immunized and LPS treated animals (N=12) and MAP-core immunized and LPS treated animals (N=15) had comparable reflexes, strength, basal muscle tone, and sensorimotor skills (Irwin, 1968; Crawley, 2000). There were no statistical differences on the accelerating rotarod (FIG. 4A; motor balance), on the plus maze and the black-white alley (FIG. 4B; tests for anxiety), or on an object-recognition memory task (FIG. 4C based on spontaneous, differential exploration of familiar and novel objects (Ennaceur and Delacour, 1988). The MAP-peptide immunized LPS treated mice however, displayed clear performance deficits on tasks thought to depend on the integrity of the hippocampus. They had enhanced locomotion when placed in a novel arena (FIG. 4D), and were impaired (indicated by chance performance) in a spatial working memory task (FIG. 4E; spontaneous alternation in T-maze (Deacib et al, 2002). In a spatial reference memory task (Morris water maze (Morris et al., 1986)), all mice showed a gradual decrease in the time taken to find the hidden platform as training proceeded (FIG. 4F, on the left). A of spatial memory, performed at the end of training, denoted probe trial, revealed that the MAP-peptide immunized mice displayed reduced spatial bias to their former target quadrant when compared to the control group (FIG. 4F, on the right, “T” columns). Because the MAP-peptide immunized mice showed a mild impairment in the standard water maze test and displayed a trend toward abnormal performance on other hippocampal-dependent tasks, we were prompted to perform a newly-designed water maze training protocol, which tests for spatial flexibility (training-to-criterion water maze (Chen et al., 2000). The MAP-peptide immunized mice displayed a significant impairment in learning this task, evidenced by their poor performance (FIG. 4G). This selective impairment in cognitive function is consistent with the neuropsychiatric studies of lupus patients that also demonstrate poor performance in verbal and spatial memory tasks (Denburg and Denburg, 1999; Brey et al., 2002).

Discussion

These data provide a model for cognitive decline in SLE. The model requires the presence of anti-NR2 antibodies found in approximately 25 to 50 percent of patients with SLE (Sharma et al., 2003), but also requires a breakdown in the blood-brain barrier. This breakdown might occur as a consequence of disease activity, such as cerebral vasculitis, but might also occur as a consequence of infection or stress and catecholaminergic excess, two conditions known to abrogate the integrity of the blood-brain barrier (Xaio et al, 2001; Abdel-Rahman et al., 2002; Esposito et al., 2002; Friedman et al., 1996). Thus, there is an explanation for the observation that cognitive decline does not parallel disease activity. One important implication of this model is that it will make it possible to test potential therapeutic interventions and obtain in vivo imaging and behavioral studies to assess their efficacy. Finally, these studies provide a model for an immunologically mediated, non-inflammatory loss of cognitive function. The presence of cognitive impairment in lupus patients who have never experienced a clinically evident inflammatory CNS insult is well documented (Leritz et al., 2000). That a systemic immune response can lead to cognitive decline, or altered mental function, without brain inflammation is likely to explain aspects of neuropsychiatric lupus. This model represents a paradigm for examining other conditions in which behavioral abnormalities result from the penetration of specific antibodies into brain tissue.

Materials and Methods

Immunization protocol. BALB/c mice, 6-8 week old females (Jackson Laboratory, Bar Harbor, Me.), were immunized intraperitoneally (I.P.) with 100 μg of antigen, either MAP-DWEYS or MAP-Core (both from AnaSpec, San Jose, Calif.), per mouse, per immunization, in 100 μL of saline. The first immunization was performed using Complete Freunds Adjuvant (CFA) and two or three boosts were given in Incomplete Freunds Adjuvant (IFA).

ELISA. ELISAs were performed as described previously (Putterman and Diamond, 1998). In brief: the plates (Costar, 3690, Corning Inc., Corning, N.Y.) were coated with DWEYS peptide at 15 μg/μL in 0.1 M NaHCO₃ (pH 8.6), o/n at 4° C. and double stranded (ds) calf thymus (CT) DNA was dry coated at 100 μg/μL, o/n at 37° C. The plates were rinsed with water and blocked with 1% BSA/PBS for 1 hr at 37° C. followed by incubation with serum at indicated dilutions in 0.2% BSA/PBS, for lhr at 37° C. After PBS-Tween wash, the secondary antibody, goat anti-mouse IgG-AP, at 1:1000 dilution in 0.2% BSA/PBS was added for 1 hr at 37° C. or o/n at 4° C. The assays were developed at RT using p-Nitrophenyl phosphate disodium salt tablets (Sigma, St. Louis, Mo.).

LPS treatment. LPS, (E. coli 055:B5, Sigma), at a dose of 3 mg/kg diluted in lactated Ringers solution to 0.6 mg/mL, was given as an I.P. injection to DWEYS or polylysine backbone immunized mice. The LPS treatment was given twice 48 hrs apart. The histology, MRS and cognitive assays were performed at indicated times after the LPS treatment.

Immunohistology. To assess IgG deposition we used anti-mouse IgG; 1:200 (Vector Laboratories Burlingame, Calif.). For detection of activated caspase-3, tissue sections were rinsed in 0.1M phosphate buffered saline (PBS, pH 7.4) for 10 min, permeabilized for 15 min in 0.2% Triton X-100 in the same buffer containing 2% bovine serum albumen (BSA), rinsed 2× in 0.1M PBS for 15 min. Non-specific binding sites were blocked with 2% BSA in 0.1M PBS for 1 hr. Sections were incubated in rabbit anti-activated caspase 3 (1:200 in blocking solution, Pharmingen, San Diego, Calif.) overnight at 4° C. Sections were rinsed 2× in 0.1M PBS, 1× in 0.1M PB for 15 min each and were then incubated for 45 min. in donkey anti-rabbit IgG-Texas Red (1:200 in 0.1M PB, Jackson Immunochemicals, West Grove, Pa.). Preparations were rinsed 3× in 0.1M PB for 45 min, mounted on gelatin-coated slides, air-dried, and coverslipped with Cytoseal (Stephens Scientific, Kalamazoo, Mich.). Final preparations were evaluated on a Zeiss Axiophot microscope fitted with an epifluorescent light source and appropriate narrow pass filter sets. Photomicrographs were composited using Adobe Photoshop.

For Fluoro-Jade staining, tissue sections were mounted onto gelatinized slides and allowed to dry at room temperature. Slides were immersed sequentially in 100% EtOH for 3 min, 70% EtOH for 1 min, dH20 for 1 min, 0.06% KMnO4 diluted in dH20 for 15 min, dH20 for 1 min, 0.001% Fluoro-Jade aqueous staining solution containing 0.1% acetic acid for 30 min (HistoChem, Jefferson, Ariz.), and rinsed 3× in dH20 for 1 min each. Preparations were dried overnight, rinsed 2× in xylene and coverslipped with CytoSeal (Stephens Scientific).

Quantitative Morphology. We modified our published techniques (Cho et al., 1997; Volpe et al., 1998) and developed an unbiased counting strategy (Coggeshall and Lekan, 1996) based on the use of stereological methods and the optical dissector (Gunderson, 1986). A 100× 100 mm frame 10 boxes on a side with its vertical axis perpendicular to the stratum pyramidale, was systematically passed along the entire length of the CA1 region. The CA1-CA2 border was identified by the change in neuron shape and packing density. All sections were viewed under oil, 100×. The counting frame was a 50 mm×100 mm subsection of the frame. Neurons were counted in the frame if part or all of the nucleus was within the frame and not in contact with the left or bottom border of the frame. For each animal, neurons in the right and left stratum pyramidale were sampled from comparable regions of the anterior dorsal hippocampus (Bregma −3.2 mm) and the posterior dorsal hippocampus (Bregma −3.8 mm). There were 12 sections from each animal and the sections separated by 160 mm. We divided the number of neurons counted by the total volume sampled to generate the density of neurons in CA1. Mean neuron density was calculated for MAP-peptide and MAP-core immunized, LPS treated animals (group), the left and right hippocampus (side), and for the anterior and posterior (regions) and for animals sacrificed after 7 and 30 days (time). Neuron density was analyzed in a four factor (group, side, region, and time) ANOVA. There was a significant effect for group [F=182.4; P<0.001]. There were no significant interactions for side, region or time. For the hippocampal volume estimates, consistent internal anatomic landmarks (for the anterior—the beginning of CA3; and for the posterior—the posterior commissure) marked the anterior—posterior border. Coronal sectioning strategy occurred as for the neuron counting and the area of the right and left hippocampus was measured digitally with K-400 programs (Zeiss, Thomwood, N.Y.). Volumes were calculated based on the product of a mean area, known tissue thickness and defined anterior—posterior region of interest. Hippocampal volume was estimated in a three factor (group, side, and time) ANOVA. There were no main effects of group. MAP-peptide and MAP-core immunized had comparable volumes [F=0.34; P=0.56), nor were there significant interactions between group and side or time.

MRSI of Mouse Brain. Spectroscopic images of the mouse brain were acquired using a 9.4 T Varian INOVA system. using a combination of an actively detunable volume coil for transmission and a 10×8 mm elliptical surface coil for reception. Animals were anesthetized and maintained on 2.5% isoflurane administered through a nose cone. The animals were placed in a plastic head frame, securing the head by teeth and ear bars.

Anatomical images were acquired using an inversion recovery sequence TR/TIR 2.9/0.9 with 11 slices (0.5 mm thick) and a 24×24 mm FOV (128×128 resolution). 3D localized volumes (1 mm×6 mm×8 mm) within the mouse brain were selected using an adiabatic refocusing method (LASER) in combination with a broad band semi-selective excitation sequence. Spectroscopic images were acquired by encoding over the selected voxel using a FOV of 24 mm×24 mm with 24×24 encodes resulting in a effective voxel size of 1 μl. The data was acquired using a TR of 2 seconds with 2 averages resulting in an acquisition time of 38 minutes. Voxels spanning the hippocampi bilaterally were selected using the anatomical images. The data was fit in the spectral domain and resonance areas used for calculations of the NAA/Cr ratio. The mean value for the hippocampal voxels was determined for each animal along with the standard deviation.

Behavioral Assays. An experimental group (MAP-peptide, n=12, MAP-peptide immunized, LPS treated) and a control group (MAP-core, n=15, MAP-core or polylysine backbone immunized, LPS treated) were studied. Mice underwent the following sequence of tests: elevated plus-maze, behavioral screen, rotarod, black-white alley, two-object recognition memory, t-maze (working memory), Morris water maze (reference memory), and training-to-criterion. The mice were run in squads of 6-10 at a time by an experimenter who was “blind” with respect to their group assignment (3-5 animals of each group in a squad).

The elevated plus maze consisted of two arms with walls (27 cm long, 8 cm wide, 30 cm high, painted black) and two open arms (30 cm long, 4 cm wide, 0.5 cm high beading, painted white). The maze was elevated 70 cm above floor level. A mouse was placed at the end of an enclosed arm and observed over a 5 min period. We measured the initial latency to enter an open arm (with the four paws), the number of entries and the total time spent in the open arms. These parameters are known to be anxiety-related (Contet et al., 2001; Deacon et al., 2002).

The behavioral screen consisted of a total of 38 separate measurements, recorded for each mouse (modified from Crawley, 2000; Irwin, 1968). Assessment started with observation in a cylindrical glass flask (height 15 cm, diameter 11 cm) followed by transfer to an arena (55×33 cm). This was continued with manipulations using tail suspension for measuring visual acuity, grip strength, body tone and reflexes. Subsequently, the mouse was restrained in supine position to record autonomic responses of skin color, limb tone and abdominal tone. Salivation and provoked biting were also recorded. The screen was completed by measuring the righting reflex and negative geotaxis. Throughout the procedure, incidences of abnormal behavior, irritability and vocalizations were recorded.

Immediately after the behavioral screen, mice were subjected to the accelerating rotarod test. Each mouse was placed in a rotating drum (ENV-576M, Med Associates Tnc, Vermont), which was accelerated from 4 to 40 rpm over the course of 5 min. The time at which each animal fell from the drum was recorded. A mouse received 3 consecutive trials and the longest time on the drum was used for analysis.

The black-white alley was adapted from (Deacon et al., 2002). Two wooden boxes (each 60×9×30 cm, with one open end, one painted black and the other white) were assembled so that their open ends faced each other, thus forming a 120 cm black-white alley. A mouse was introduced in the black end and was left in the alley for 1 min. We scored the initial latency to cross into the white side, the number of crossings (four paws), and the time spent in the white side.

Two-object recognition memory was tested in a chamber (25 cm long, 25 cm wide, 60 cm high) with open ceiling, constructed of white plastic. It was illuminated from the top by a 40 W white bulb. A CCD camera was also mounted on top and was used for videotaping behavior. The floor was covered with a thin layer of bedding. Before formal testing, each mouse was allowed to familiarize with the empty chamber for 3 sessions (5 minutes each over a period of 2 days). A single trial consisted of 3 phases: sample, delay, and choice. For the sample phase, mice were placed in the chamber and allowed to explore two identical objects for a period of 5 min. For the delay phase, mice were placed in their home cage for 10 min. During this interval, test objects replaced sample objects. One of them was identical to those in the sample phase (“familiar object”) whereas the other was different (“novel object”). Extreme care was taken in placing the objects in exactly the same positions occupied by the sample objects. For the choice phase, mice were allowed to explore for 5 min. Object exploration was scored when the mouse touched the object with the face (mouth, whiskers and nose). Touching the object with any other part of the body while facing another direction was not counted as an exploration. After the experiment, tapes of the trials were revised and the exact time spent on each of the objects was obtained. We defined A1 and A2 as the times exploring the sample objects, A3 as the time exploring the familiar object, and B1 as the time exploring the novel object. Various parameters were calculated in order to examine recognition memory: E1, proportion of exploration in the sample phase, defined as [(A1+A2)/300]; E2, proportion of exploration in the test phase, defined as [(A3+B1)/300]; D1, preference index, defined as [B1−A3], and D2, discrimination ratio, defined as [(B1−A3)/(B1+A3)].

The T-maze task was adapted from (Deacon et al., 2002). The apparatus consisted of an enclosed T-maze, each arm was 30 cm long×10 cm wide×29 cm high, made of white painted plastic. The side walls were not perpendicular but angled at 110 degrees (towards the outside). A removable central partition extended 7 cm from the back of the T into the start arm, dividing the choice area. A thin layer of bedding was placed on the floor. In the first phase of the test the central partition was in place and the mouse navigated from the end of the start arm. After it entered a goal arm it was confined there for 30 s (by blocking the entrance), before being returned to the start arm by the experimenter. During the delay, the central partition was removed. The second phase consisted in the mouse simply choosing between the two arms. The choice arm was the one the animal first entered with the whole body. Each mouse received 16 trials over 4 days; the minimum inter-trial interval was 1 h.

The Morris water maze was used to test for spatial reference memory (Morris et al., 1986). The apparatus consisted of a circular pool (160 cm diameter, opaque water at 20±1° C.) surrounded by prominent visual cues. Mice were placed into the water facing the side walls and allowed to swim until they found the hidden platform (top surface 1.5 cm below water level). The maximum trial duration was 90 s, with 20 s on platform at end of trials. Swim paths were monitored by video. The task was divided into two phases: in the first, mice were trained to find a “large” hidden platform (diameter, 24 cm) for 12 trials (4 trials per day). After this, a first probe trial was performed: each mouse swam for 60 s with the platform removed from the pool. The second phase consisted of 24 trials, in which the mice found a “small” platform (diameter, 16 cm). A second probe trial ensued at the end of training.

For the training-to-criterion task, mice were required to find five consecutive locations in the water maze. Each animal was trained, for up to eight trials per day, to a performance criterion of three successive trials with an escape latency of less than 20 s before being transferred to the next location on the next day (maximum trials was 40 for location 1, and 32 trials thereafter). For each mouse, the platform was moved between different locations, drawn from a set of 20 possible locations.

### EXAMPLE 2

### Human Histological Studies

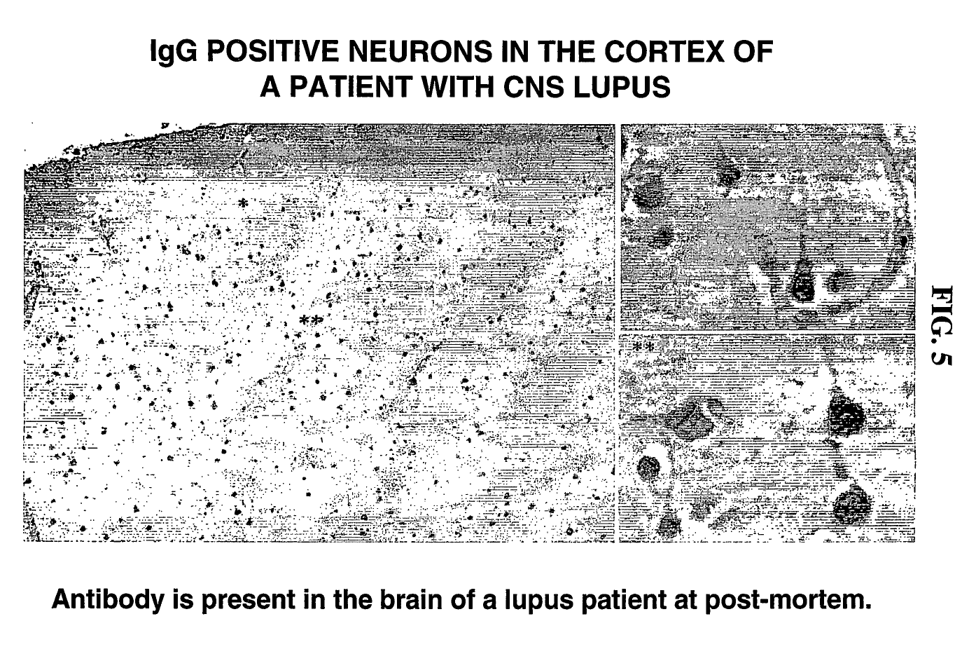

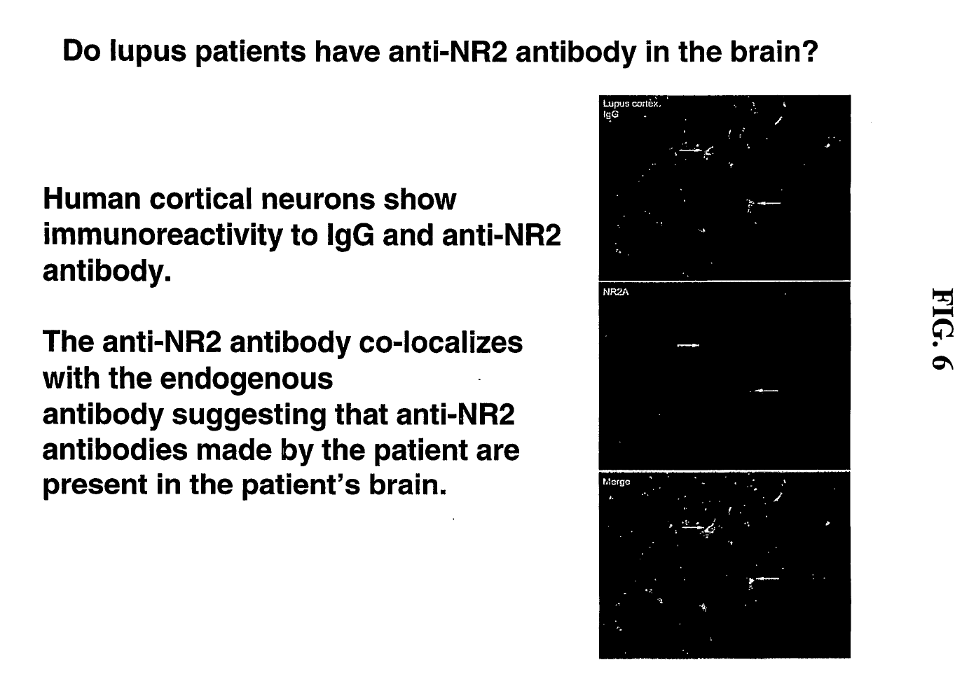

Further histological studies utilized a post-mortem lupus patient. Sections from the patient were immunostained for IgG. Neurons stained positively for IgG (FIG. 5), showing that IgG did cross the blood-brain barrier and bound to neurons. Additionally, anti-NR2 antibody colocalized with anti-IgG antibody (FIG. 6). Similar observations have been made in 4 other SLE patients but not in patients with other cognitive diseases.

In view of the above, it will be seen that the several advantages of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

1. A method of preventing binding of an anti-double stranded (ds)-DNA antibody to a neuron in a mammal exhibiting or at risk for lupus-induced cognitive dysfunction, wherein the anti-ds-DNA antibody binds to an NR2 subunit of an NMDA receptor on the neuron, the method comprising treating the mammal with at least one peptide or mimetic in an amount effective to bind to the antibody, wherein the peptide or mimetic comprises an amino acid sequence of X1-Trp-X1-Tyr-X2, wherein X1 represents Asp or Glu, and X2 represents Gly or Ser.

2. The method of claim 1, wherein the peptide or mimetic comprises D-amino acids.

3. The method of claim 1, wherein the peptide or mimetic is 5-30 amino acids in length.

4. (canceled)

5. The method of claim 1, wherein the peptide or mimetic is 5 amino acids in length.

6. The method of claim 1, wherein the peptide or mimetic comprises Asp-Trp-Glu-Tyr-Ser.

7-8. (canceled)

9. The method of claim 1, wherein the neuron is a hippocampal neuron.

10-20. (canceled)

21. The method of claim 1, wherein the risk for lupus-induced cognitive dysfunction is determined by determining whether the mammal has anti-NR2 antibodies, wherein the presence of anti-NR2 antibodies indicates that the mammal is at risk for lupus-induced cognitive dysfunction.

22. The method of claim 21, wherein the cerebrospinal fluid is tested for the presence of anti-NR2 antibodies.

23. A method of inhibiting progression of cognitive dysfunction in a mammal exhibiting or at risk for lupus-induced cognitive dysfunction, the method comprising treating the mammal with an agent that prevents binding of an anti-ds-DNA antibody to an NR2 subunit of an NMDA receptor of a neuron.

24. The method of claim 23, wherein the agent is an antibody or an aptamer that binds to the NMDA receptor on a neuron but does not induce neuronal death.

25. The method of claim 23, wherein the neuron is in the hippocampus.

26-43. (canceled)

44. The method of claim 1, wherein the peptide or mimetic is administered to the brain of the mammal.

45. The method of claim 23, wherein the agent is administered to the brain of the mammal.

46. A method of determining whether a patient is at risk for lupus-induced cognitive dysfunction, the method comprising determining whether the patient has anti-NR2 antibodies, wherein the presence of anti-NR2 antibodies indicates that the patient is at risk for lupus-induced cognitive dysfunction.

47. The method of claim 46, wherein the cerebrospinal fluid is tested for the presence of anti-NR2 antibodies.

48. The method of claim 46, wherein the peripheral blood is tested for the presence of anti-NR2 antibodies.

49. The method of claim 46, the method further comprising treating the patient with an agent that prevents binding of an anti-NR2 antibody to an NR2 subunit of an NMDA receptor of a neuron if the patient is at risk for lupus-induced cognitive dysfunction.

50. The method of claim 49, wherein the agent is at least one peptide or mimetic that comprises an amino acid sequence of X1-Trp-X1-Tyr-X2, wherein X1 represents Asp or Glu, and X2 represents Gly or Ser.

51. The method of claim 50, wherein the peptide or mimetic comprises Asp-Trp-Glu-Tyr-Ser.

52. The method of claim 46, the method further comprising treating the patient with an agent that inhibits death of a neuron that comprises a bound anti-ds-DNA antibody on an NR2 subunit of an NMDA receptor on the neuron if the patient is at risk for lupus-induced cognitive dysfunction.
